SEQUENCE LISTING (12 sequences)

SEQ ID NO: 1 - protein, 10 aa, Homo sapiens
  misc-feature (1)..(1): Xaa is pyroGlu
  misc-feature (10)..(10): Xaa is Gly-NH2
XHWSYGLRPX

SEQ ID NO: 2 - protein, 9 aa, Artificial Sequence
  Synthetic Sequence
  misc-feature (1)..(1): Xaa is pyroGlu
  misc-feature (6)..(6): Xaa is D-Leu
  misc-feature (9)..(9): Xaa is Pro-NHEt
XHWSYXLRX

SEQ ID NO: 3 - protein, 9 aa, Artificial Sequence
  Synthetic
  misc-feature (1)..(1): Xaa is pyroGlu
  misc-feature (6)..(6): Xaa is D-Ser(tBu)
  misc-feature (9)..(9): Xaa is Pro-NHEt
XHWSYXLRX

SEQ ID NO: 4 - protein, 9 aa, Artificial Sequence
  Synthetic Sequence
  misc-feature (1)..(1): Xaa is Oxo-Pro
  misc-feature (6)..(6): Xaa is Ntbenzyl-D-His
  misc-feature (9)..(9): Xaa is N-Et-L-prolinamide
XHWSYXLRX

SEQ ID NO: 5 - protein, 10 aa, Artificial Sequence
  Synthetic Sequence
  misc-feature (1)..(1): Xaa is pyroGlu
  misc-feature (6)..(6): Xaa is D-Ser(tBu)
  misc-feature (10)..(10): Xaa is AzaGly-NH2
XHWSYXLRPX

SEQ ID NO: 6 - protein, 9 aa, Artificial Sequence
  Synthetic Sequence
  misc-feature (1)..(1): Xaa is pyroGlu
  misc-feature (6)..(6): Xaa is D-Trp
  misc-feature (9)..(9): Xaa is Pro-NHEt
XHWSYXLRX

SEQ ID NO: 7 - protein, 10 aa, Artificial Sequence
  Synthetic Sequence
  misc-feature (1)..(1): Xaa is Oxo-Pro
  misc-feature (6)..(6): Xaa is 3-(2-naphtyl)-D-Ala
  misc-feature (10)..(10): Xaa is Gly-NH2
XHWSYXLRPX

SEQ ID NO: 8 - protein, 10 aa, Artificial Sequence
  Synthetic Sequence
  misc-feature (1)..(1): Xaa is pyroGlu
  misc-feature (6)..(6): Xaa is D-Trp
  misc-feature (10)..(10): Xaa is Gly-NH2
XHWSYXLRPX

SEQ ID NO: 9 - protein, 10 aa, Artificial Sequence
  Synthetic Sequence
  misc-feature (1)..(1): Xaa is N-acetyl-3-(2-naphthyl)-D-alanyl
  misc-feature (2)..(2): Xaa is 4-chloro-D-phenylalanyl
  misc-feature (3)..(3): Xaa is 3-(3-pyridyl)-D-alanyl
  misc-feature (6)..(6): Xaa is D-Cit
  misc-feature (10)..(10): Xaa is D-Ala-NH2
XXXSYXLRPX

SEQ ID NO: 10 - protein, 10 aa, Artificial Sequence
  Synthetic Sequence
  misc-feature (1)..(1): Xaa is N-acetyl-3-(2-naphthyl)-D-alanyl
  misc-feature (2)..(2): Xaa is 4-chloro-D-phenylalanyl
  misc-feature (3)..(3): Xaa is 3-(3-pyridyl)-D-alanyl
  misc-feature (5)..(5): Xaa is N(Me)Tyr
  misc-feature (6)..(6): Xaa is D-Asn
  misc-feature (8)..(8): Xaa is N6-isopropyl-L-lysyl
  misc-feature (10)..(10): Xaa is D-Ala-NH2
XXXSXXLXPX

SEQ ID NO: 11 - protein, 10 aa, Artificial Sequence
  Synthetic Sequence
  misc-feature (1)..(1): Xaa is N-acetyl-3-(2-naphthyl)-D-alanyl
  misc-feature (2)..(2): Xaa is 4-chloro-D-phenylalanyl
  misc-feature (3)..(3): Xaa is 3-(3-pyridyl)-D-alanyl
  misc-feature (5)..(5): Xaa is 4Aph(L-Hor)
  misc-feature (6)..(6): Xaa is D-4Aph(Cbm)
  misc-feature (8)..(8): Xaa is N6-isopropyl-L-lysyl
  misc-feature (10)..(10): Xaa is D-Ala-NH2
XXXSXXLXPX

SEQ ID NO: 12 - protein, 10 aa, Artificial Sequence
  Synthetic Sequence
  misc-feature (1)..(1): Xaa is N-acetyl-3-(2-naphthyl)-D-alanyl
  misc-feature (2)..(2): Xaa is 4-chloro-D-phenylalanyl
  misc-feature (3)..(3): Xaa is 3-(3-pyridyl)-D-alanyl
  misc-feature (6)..(6): Xaa is D-hArg(Et2)
  misc-feature (8)..(8): Xaa is hArg(Et2)
  misc-feature (10)..(10): Xaa is D-Ala-NH2
XXXSYXLXPX

(19) United States
(12) Patent Application Publication
(10) Pub. No.: US 2022/0347260 A1
(43) Pub. Date: Nov. 3, 2022

(54) Combination of An Agonist Anti-PD-1 Antibody With a GnRH Agonist or Antagonist to Treat Cancer

(71) Applicant: Debiopharm International SA, Lausanne (CH)

(72) Inventors: Ferdinand LEJEUNE, Lausanne (CH); Bruno GAVILLET, Mourex (FR)

(21) Appl. No.: 16/954,042

(22) PCT Filed: Dec. 18, 2018
(86) PCT No.: PCT/IB2018/001445

§ 371 Date: Jun. 15, 2020

Related U.S. Application Data
(63) Continuation of application No. PCT/IB2017/001576, filed on Dec. 18, 2017.

(51) Int. Cl. A61K 38/09; A61P 35/00; A61K 31/427; A61K 31/47

(52) U.S. Cl. CPC A61K 38/09; A61P 35/00; A61K 31/427; A61K 31/47

(57) ABSTRACT

The present disclosure relates to a novel method of treatment of a cancer patient in which the patient is subjected to both an inhibitor of an immune check point molecule, preferably “Programmed Death 1” (PD-1) or its ligand “programmed death ligand 1” (PD-L1), and a Gonadotropin-Releasing Hormone (GnRH, also known as LHRH or FSH-RH) agonist or antagonist.

### FIELD OF THE INVENTION

The present disclosure relates to a novel method of treatment of a cancer patient in which the patient is subjected to both an inhibitor of an immune check point molecule, preferably “Programmed Death 1” (PD-1) or its ligand “programmed death ligand 1” (PD-L1), and a Gonadotropin-Releasing Hormone (GnRH, also known as LHRH or FSH-RH) agonist or antagonist.

### BACKGROUND OF THE INVENTION

Typically, immune destruction of tumor cells is inefficient. It now appears that this is not because cancer patients do not have a significant reservoir of T cells capable of destroying their tumor, but because cells of the adaptive and innate immune systems are held in check or are neutralized by pathways that inhibit their activation or their effector functions. Instrumental in this suppression are so-called immune checkpoint molecules. Several such checkpoint molecules have been identified over the last twenty years. The prototypical molecule of this type is the cytotoxic T lymphocyte antigen 4 (CTLA-4). Blocking this molecule was found to result in tumor regression in murine models. Leach et al. (1996) Science 271: 1734-1736. CTLA-4 is expressed on activated T cells, predominantly on CD4+ cells, and limits T cell responses by interfering with the activity of master T cell co-stimulator CD28. CTLA-4 and CD28 share ligands CD80 and CD86, whereby CTLA-4 outcompetes CD28 due to its higher affinity for the latter ligands. Linsley et al. (1994) Immunity 1: 793-801. In addition to this competition for ligand binding, CTLA-4 may also cause ligand depletion by trans-endocytosis. It appears that CTLA-4 counteracts both tyrosine and serine/threonine kinase signals induced by T cell receptor (TCR) and CD28 by activating phosphatases SHP2 and PP2A. Rudd et al. (2009) Immunol. Rev. 229: 12-26. It also interferes with the extended interaction between TCR and peptide-MHC. Schneider et al. (2006) Science 313: 1972-1975. The immune checkpoint molecule plays a key role in maintaining immune tolerance as shown in mice deficient in CTLA-4. Tivol et al. (1995) Immunity 3: 541-547; Waterhouse et al. (1995) Science 270: 985-988. The correlate in humans is a high rate of inflammatory side effects upon inhibition of CTLA-4. Phan et al. (2003) Proc. Natl. Acad. Sci. USA 100: 8372-8377.

Two monoclonal CTLA-4-blocking antibodies have been undergoing clinical development. One is ipilimumab, a fully human IgG1 antibody from Bristol-Myers Squibb. The other is tremelimumab, a fully human IgG2 antibody from Pfizer/Medimmune. It is noted that ipilimumab may not only block CTLA-4 activity but also cause depletion of CTLA-4-expressing regulatory T cells (Treg). Ipilimumab has been approved in the U.S., Canada and Europe for the treatment of unresectable or metastatic melanoma. It is also indicated for adjuvant treatment of stage III patients. In a meta-analysis of melanoma patients that had been enrolled in phase II or III trials, Ipilimumab treatment resulted in durable survival in about 20% of patients, in some cases for more than ten years. Schadendorf et al. (2015) J. Clin. Oncol. 33: 1889-1894. Anti-tumor effects were substantially more modest for NSCLC and RCC.

Like CTLA-4, immune checkpoint molecule PD-1 is expressed on activated T cells. Parry et al. (2005) Mol. Cell. Biol. 25: 9543-9553. It also activates phosphatases SHP2 and PP2A. Engagement of PD-1 is thought to directly interfere with TCR-mediated effector functions and increase T cell migration. The checkpoint molecule is believed to exert its function primarily in the tumor microenvironment, whereas CTLA-4 acts primarily in secondary lymphoid tissues. Wing et al. (2008) Science 322: 271-275; Peggs et al. (2009) J. Exp. Med. 206: 1717-1725. The two known ligands of PD-1 are PD-L1 and PD-L2. Dong et al. (1999) Nat. Med. 5: 1365-1369; Latchman et al. (2001) Nat. Immunol. 261-268; Tseng et al. (2001) J. Exp. Med. 193: 839-846. The ligand molecules share homology but are divergently regulated. PD-L1 is induced in activated hematopoietic and epithelial cells by interferon-y (produced by activated T cells and natural killer cells) and is expressed constitutively by certain tumor cells. Tumeh et al. (2014) Nature 515: 568-571. PD-L2 is found induced in activated dendritic cells and some macrophages. Induction may be predominantly by IL-4. PD-1 knockout mice exhibit late-onset organ-specific inflammation. Nishimura et al. (1999) Immunity 11: 141-151; Science 291: 319-322 (2001). This phenotype is much less severe than that observed in CTLA-4 knockout mice. Correspondingly, clinical immune-related effects of anti-PD-1 therapy tend to be milder than those associated with anti-CTLA-4 therapy. PD-L1 is expressed in many solid tumors, and PD-L2 in certain subsets of B cell lymphomas. PD-1 is highly expressed in tumor-infiltrating lymphocytes. Dong et al. (2002) Nat. Med. 8: 793-800; Ansell et al. (2015) N. Engl. J. Med. 372: 311-319; Amadzadeh et al. (2009) Blood 114: 1537-1544; Sfanos et al. (2009) Prostate 69: 1694-1703.

The first human trials of anti-PD-1 therapy employed monoclonal antibody nivolumab, a fully human IgG4 antibody from Bristol-Myers Squibb/Ono Pharmaceuticals. Objective response rates of 17% for advanced treatment-refractory NSCLC, 20% for RCC and 31% for melanoma were documented. Many of these responses were durable. Overall survival was 9.9, 22.4 and 16.8 months, respectively. Topalian et al. (2012) N. Engl. J. Med. 366: 2443-2454; J. Clin. Oncol. 32: 1020-1030 (2014). Nivolumab has been approved in the U.S., Japan and Europe for the treatment of unresectable or metastatic melanoma. It is also indicated for metastatic non-small cell lung carcinoma (NSCLC) that failed platinum therapy and advanced renal cell carcinoma (RCC) subsequent to anti-angiogenic therapy. It is noted that durable responses have also been observed in head and neck and bladder cancer patients treated with nivolumab. Monoclonal anti-PD-1 antibody pembrolizumab, a humanized IgG4 antibody from Merck has also been approved for advanced melanoma and NSCLC indications. Atezolizumab, another antibody of the IgG1 type from Roche/Genentech, inhibits the ligand PD-L1. It obtained accelerated FDA approval for locally advanced or metastatic urothelial carcinoma treatment after failure of chemo or radiotherapy. It is noted that multiple antibodies directed to PD-L1 (BMS-936559, MEDI4736, MPDL3280A, MSB0010718C) are moving through clinical trials. As of 2015, objective response rates for anti-PD-1/PD-L1 therapies have been reported to be 17-40% for melanoma, 10-30% for lung cancer, 12-29% for kidney cancer, 25% for bladder cancer, 6-23% for ovarian cancer, 14-20% for head and neck cancer, 22% for gastric cancer, 24% for colorectal cancer, 18% for triple-negative breast cancer, 24% for mesothelioma and 87% for Hodgkin's lymphoma. Lejeune (2015) Melanoma Res. 25: 373-375. The term “anti-PD-1 molecule” as used herein refers to anti-PD-1 and/or anti-PD-L1 inhibitors. More particularly, the term “anti-PD-1 molecule” relates to inhibitory anti-PD-1 or anti-PD-L1 antibodies.

Work with animal models had suggested that combination of anti-CTLA-4 and anti-PD-1 therapies may have synergistic effects. Ongoing clinical studies have found early and substantial regressions for an ipilimumab/nivolumab combination. Unfortunately, immune-related adverse reactions were also enhanced compared to those provoked by the drugs administered singly. Wolchok et al. (2013) N. Engl. J. Med. 369: 122-133.

The structure of Gonadotropin-Releasing Hormone (GnRH; also known as LHRH or FSH-RH) is pyroGlu-His-Trp-Ser-Tyr-Gly-Leu-Arg-Pro-Gly-NH2, whereby “pyroGlu” refers to pyroglutamate and “Gly-NH2” to 2-amino-acetamide. Matsuo et al. (1971) Biophys. Biochem. Res. Commun. 43: 1334-1339; Burgus et al. (1972) Proc. Natl. Acad. Sci. USA 69: 278-282. The decapeptide is synthesized in the hypothalamus and then released into the hypophysal portal blood stream. At the anterior pituitary, GnRH stimulates the synthesis and secretion of gonadotropins Follicle-Stimulating Hormone (FSH) and Luteinizing Hormone (LH). The latter processes are controlled by the intensity and frequency of GnRH pulses and are feedback-controlled by androgens and estrogens. GnRH agonists (also referred to herein as GnRH-A) are synthetic compounds modeled after the natural GnRH with specific modifications, typically in position 6 (amino acid substitution), 9 (alkylation) and 10 (deletion). These modifications inhibit rapid degradation. The structures of the peptides/peptide salts are shown below.

Leuprolide:
pyroGlu-His-Trp-Ser-Tyr-D-Leu-Leu-Arg-Pro-NHEt
Buserelin:
pyroGlu-His-Trp-Ser-Tyr-D-Ser(tBu)-Leu-Arg-Pro-
NHEt
Histrelin:
Oxo-Pro-His-Trp-Ser-Tyr-Ntbenzyl-D-His-Leu-Arg-
N-Et-L-prolinamide
Goserelin:
pyroGlu-His-Trp-Ser-Tyr-D-Ser(tBu)-Leu-Arg-Pro-
Azagly-NH₂
Deslorelin:
pyroGlu-His-Trp-Ser-Tyr-D-Trp-Leu-Arg-Pro-NHEt
Nafarelin:
Oxo-Pro-His-Trp-Ser-Tyr-3-(2-naphtyl)-D-Ala-
Leu-Arg-Pro-Gly-NH₂
Triptorelin:
pyroGlu-His-Trp-Ser-Tyr-D-Trp-Leu-Arg-Pro-Gly-
NH₂

Agonists do not quickly dissociate from the GnRH receptor. As a result, GnRH-A administration produces an initial increase in FSH and LH secretion. After about ten days, a profound hypogonadotrophical effect (i.e. decrease in FSH and LH) occurs through pituitary GnRH receptor downregulation by internalization of receptors, resulting in hypogonadism, i.e. a reversible functional deficiency of androgen and estrogen. Common side effects of GnRH-A therapy are hot flashes, headaches, decreased libido and erectile dysfunction.

Human peripheral blood lymphocytes were found to produce LH when exposed to GnRH, implying the presence of a GnRH receptor on the cells. Ebaugh & Smith (1987). Fed. Proc. 46: 7811. A confirmatory study on mouse splenocytes used a GnRH receptor antibody to demonstrate the presence of a similar receptor on the lymphocytes. Costa et al. (1990) Prog. NeuroEndocrinlmmunology 3: 55-60. IL-2 receptor expression in splenocytes and thymocytes isolated from (female) rats could be induced by GnRH or GNRH-A under both basal and stimulated conditions. Batticane et al. (1991) Endocrinol. 129: 277-286. The presence of a high-affinity GnRH receptor in membranes of cultured porcine lymphocytes was demonstrated by a radioreceptor assay using a GnRH-A as the labeled/unlabeled ligand. Standaert et al. (1992) Biology of Reproduction 46; 997-1000. See also Marchetti et al. (1989) Endocrinol. 125: 1025-1036. Human peripheral blood mononuclear cells were found to express GnRH, GnRH receptor and IL-2 receptor mRNAs. In vitro, GnRH and GNRH-A enhanced the expression of GnRH receptor mRNA, and GnRH of IL-2 receptor mRNA, suggesting that lymphocyte-produced GnRH may act as an autocrine or paracrine factor to regulate immune function. Chen et al. (1999) J. Clin. Endocrin. Metab. 84: 743-750. Stimulation of IL-2 receptor mRNA expression in human lymphocytes was confirmed by Tanriverdi et al. (Clin. Exp. Immunol. 142: 103-110 (2005)).

GnRH and/or GnRH-A enhance lymphocyte proliferation stimulated by mitogen or cytokine in splenocytes and thymocytes from mice (Batticane et al. (1991)), thymocytes from old rats (GnRH-A was administered in vivo; Marchetti et al. (1989)) and human lymphocytes (Tanriverdi et al. (2005)). There are in vivo correlates of these findings. The process of aging is accompanied by thymus involution and a parallel decrease in GnRH-A binding sites in both male and female rats. Marchetti et al. (1989). Chronic treatment with GnRH-A largely reversed this decline. Orchidectomy had a somewhat smaller effect. Largest effects were seen when orchidectomy and GnRH-A treatment were combined. Infusions of GnRH-A in pregnant rats reduced pregnancy-induced involution, resulting in a significant increase in thymus weight and thymocyte numbers. Dixit et al. (2003) Endocrinol. 144: 1496-1505. In a different study, lymphocytes isolated from 24-h GnRH-A-treated or mock-treated pregnant rats were stimulated by plated anti-CD3. Dixit et al. (2003) Biology of Reproduction 68: 2215-2221. A marked increase in IFNγ and an inhibition of IL-4 production were measured in lymphocytes from the GnRH-A-treated animals. Analogous results were obtained when lymphocytes were GnRH-A-treated in vitro. These results indicate that GnRH-A can function as a Th1 inducer and a Th2 inhibitor.

While some of the above-described effects of GnRH-A would appear to be direct consequences of activation of GnRH receptors on lymphocytes, GnRH-A may also affect the immune system indirectly, as a consequence of the reduction in sex hormone levels that they cause.

Estrogen has a highly pleiotropic effect on the immune system. Essentially all facets of the immune response are affected in one way or another. Of particular interest may be that estrogen promotes the expansion and activation of Treg cells via ERα-mediated signaling. Polanczyk et al. (2004) J. Immunol. 173: 2227-2230; Tai et al. (2008) J. Cell. Physiol. 214: 456-64. Estrogen also upregulates Th2 cytokine IL-4 on stimulated CD4+ T lymphocytes, suppressing MHC class II expression, rendering the CD4+ cells ignorant of tumor antigens. Lambert et al. (2005) J. Immunol. 175: 5716-5723. LHRH-A downregulated CD4+CD25+ (Tregs) in women. Ho et al. (1995) American Journal of Reproductive Immunology 33: 243-252.

Testosterone has been implicated as a negative regulator of the immune response to pathogens and cancer. CD4+ T cells isolated from spleens of male mice differentiated under Th1-polarizing conditions in the presence of an androgen produced less IFNγ and T-bet than when differentiated in the absence of the hormone. This finding suggested that androgen exerts a negative effect during an early event involved in Th1 differentiation. Kissick et al. (2014) Proc. Natl. Acad. Sci. USA 111: 9887-9892. Androgen was found to inhibit Th1 differentiation by inhibiting IL12-induced Stat4 phosphorylation. An interaction between androgen receptor and tyrosine phosphatase Ptpn1 results in upregulation of Ptpn1 expression and a consequential inhibition of IL-12 signaling in CD4+ T cells. The relevance of this mechanism in human biology was suggested by the observation that the level of Ptpn1 was significantly lower in CD4+ T cells from prostate cancer patients treated with androgen deprivation therapy than in cells from control patients. The latter mechanism may result in T cell anergy or induction of Treg cells.

Androgen deprivation increases lymphocyte infiltration of the prostate in both mice and men. Roden et al. (2004) J. Immunol. 173: 6098-6108; Mercader et al. (2001) Proc. Natl. Acad. Sci. USA 98(25): 14565-14570. More recently it was found in experiments with mice that castration resulted in T cell infiltration not only of the prostate but also of the lung and the intestine. Kissick et al. (2014).

Surgical castration and chemical castration by GnRH-A have profound and durable effects on the function of T lymphocytes. At puberty, an androgen burst induces thymus involution resulting in progressive downregulation of the immune system, a condition of T cell pool depletion.

Androgen ablation by GnRH-A in an adult male mouse or a rat quickly regenerates the thymus and its function in terms of proliferative response to mitogens. Marchetti et al. (1989); Sutherland et al. (2005) J. Immunol. 175: 2741-2753. Prostate cancer patients treated with GnRH-A showed the apparition in the blood of the thymus marker TREC (T cell Receptor Excision Circle), a finding strongly suggesting that the thymus in the aged men was induced to regenerate. Sutherland et al. (2005). In patients receiving stem cells transplants, GnRH-A increased TRECs in men and women (Sutherland et al. (2008) Clin. Cancer Res. 14: 1138-1149.

In male mice, castration increased CD4+ and CD8+ lymphocyte numbers in the circulation as well as in peripheral lymph nodes. Roden et al. (2004) J. Immunol. 173: 6098-6108. In prostate cancer patients, a combination of GnRH-A and anti-androgen (flutamide) mitigated lymphopenia due to radiotherapy. (Johnke et al. (2005) Anticancer Res. 25: 3159-3166. Four months after receiving GnRH-A, prostate cancer patients had increased circulating CD4+ and CD8+ T lymphocytes, increased naïve and memory T cells as well as increased NK cells. Sutherland et al. (2005).

Already after 7 days, GnRH-A (leuprolide 1-month formulation) combined with flutamide augmented tumor-infiltrating lymphocytes in prostate tumors (Mercader M. 2001), an early effect that likely is immunological and can be caused by a thymus rejuvenation together with an immune response against apoptotic tumor cells resulting from androgen depletion. Cytotoxic CD8+T lymphocytes expressing granzyme accumulated in the prostate cancer stroma of mice 2.5 weeks after castration. Akins et al. (2010) Cancer Res. 70: 3473-3482. In patients receiving stem cells transplants, GnRH-A extended the Vbeta repertoire of CD4+ and CD8+ receptors and stimulated CD8+ T cell proliferation upon stimulation with anti-CD-3 and anti-CD-28. Sutherland et al. (2008).

GnRH antagonists (also referred to herein as GnRH-At) are either synthetic peptide derivatives of the natural GnRH or non-peptidic small molecules. The known structures are shown below:

### Cetrorelix: [N-Ac-D-Nal(2)1, D-Phe(4Cl)2, D-Pal(3)3, D-Cit6, D-Ala10]LHRH

### Abarelix: [N-Ac-D-Nal(2)1, D-Phe(4Cl)2, D-Pal(3)3, NMe-Tyr5, D-Asn6, ILys8-D-Ala10]LHRH

### Degarelix: [N-Ac-D-Nal(2)1, D-Phe(4Cl)2-D-Pal(3)3, 4Aph(L-Hor)5, D-4Aph(Cbm)6, Lys8(iPr), D-Ala10]LHRH

### Ganirelix: [N-Ac-D-Nal(2)1, D-Phe(4Cl)2, D-Pal(3)3, D-hArg(Et2)6, L-hArg(Et2)8, D-Ala10]LHRH

In the above formulas : Ac, acetyl group; Nal(2), 2-Naphthylalanine; Phe(4Cl) or 4CPa, 4-Chlorophenylalanine; Pal(3), 3-Pyridylalanine; Cit, citrulline; LHRH, luteinizing hormonereleasing hormone; NM, N-methyl; ILys, Ne-isopropyllisine; Hor, hydroorotyl; Lys(iPr), N6-lsopropyllysine; 4Aph, 4-Aminophenylalanine; Cbm,carbamoyl group; hArg, homoarginine.

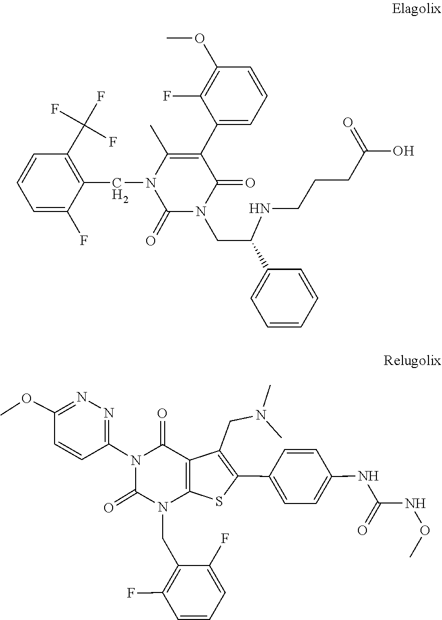 [SMILES: COc1cccc(-c2c(C)n(Cc3c(F)cccc3C(F)(F)F)c(=O)n(C[C@H](NCCCC(=O)O)c3ccccc3)c2=O)c1F ; CONC(=O)Nc1ccc(-c2sc3c(c2CN(C)C)c(=O)n(-c2ccc(OC)nn2)c(=O)n3Cc2c(F)cccc2F)cc1]

Treatment with GnRH antagonists decreases or blocks GnRH action in the body: GnRH antagonists compete with natural GnRH for binding to GnRH receptors in the pituitary gland, causing an immediate reversible suppression of gonadotropin secretion (without initial hypersecretion of gonadotropins and initial increase in serum testosterone/estrogen). The resulting androgen/estrogen deprivation may produce an immunostimulating effect similar to that of GnRH agonists.

Even in indications that are thought to be most susceptible to therapy with an anti-PD-1 antibody such as nivolumab or pembrolizumab, the therapy is not producing effective responses in the majority of patients. To improve efficacy, combinations of two inhibitors targeting different immune checkpoint molecules, e.g., nivolumab and ipilimumab, have been investigated. As already mentioned before, such combination therapy produces severe immune-related side effects in a substantial fraction of patients.

Thus, there is still a need for improved cancer therapies. In particular, there is a need for methods of treating cancer that improve the efficacy of anti-PD1 molecules in one or more types of cancer. There is also a need for improved cancer therapies for patients who have been found refractory or have become resistant to a treatment including an anti-PD1 molecule. The present invention provides a combination for use in the treatment of cancer to address the above needs.

### SUMMARY OF THE INVENTION

The present disclosure relates to a method of treatment of a human patient attained by a cancer. The method comprises administering to the patient an effective dose of an anti-PD-1 molecule in association with an effective dose of a GnRH agonist or a GnRH antagonist. The term “anti-PD-1 molecule” is used to designate anti-PD-1 and/or anti-PD-L1 inhibitors. Without wishing to be bond to any theory, the GnRH-A or GnRH-At is added to modify the immune system of the cancer patient, resulting in increasing the potential targets of the anti-PD-1 molecule (in particular the tumor infiltrating lymphocytes — TILs). As a consequence, the combination of GnRH-A or GnRH-At and anti-PD-1 molecule is expected to result in a better therapeutic effect compared with the individual active agents, without causing the severe immune-related toxicity that combinations of immune checkpoint inhibitors produce.

In a particular embodiment, the present disclosure relates to the use of an anti-PD-1 molecule in association with a GnRH agonist in the manufacture of a medicament for the treatment of a cancer in a human patient, or to an anti-PD-1 molecule in association with a GnRH agonist for use in the treatment of a cancer in a human patient. The claimed treatments involve combinations of an anti-PD-1 molecule and a GnRH agonist. The term “in association with” is used to indicate that the GnRH agonist can be administered prior to, concurrently with, or after administration of the anti-PD-1 molecule. Within a therapeutic regimen, one of the active agents may be administered at shorter intervals than the other, and the agents can be administered by different routes.

In another particular embodiment, the present disclosure also relates to the use of an anti-PD-1 molecule in association with a GnRH antagonist in the manufacture of a medicament for the treatment of a cancer in a human patient, or to an anti-PD-1 molecule in association with a GnRH antagonist for use in the treatment of a cancer in a human patient. The claimed treatments involve combinations of an anti-PD-1 molecule and a GnRH antagonist. The term “in association with” is used to indicate that the GnRH antagonist can be administered prior to, concurrently with, or after administration of the anti-PD-1 molecule. Within a therapeutic regimen, one of the active agents may be administered at shorter intervals than the other, and the agents can be administered by different routes.

In a particular embodiment, the cancer with which the patient is afflicted is any cancer but not a prostate cancer. In more particular embodiments, the patient's cancer is a melanoma, a lung cancer, a kidney cancer, a bladder cancer, an ovarian cancer, a head and neck cancer, a gastric cancer, a colorectal cancer, a triple-negative breast cancer, a mesothelioma or a Hodgkin's lymphoma. In a specific embodiment, the cancer is a melanoma.

The anti-PD-1 molecule administered is an inhibitor of the function of immune checkpoint molecule PD-1 or its ligand PD-L1. In particular embodiments, the inhibitor is an anti-PD-1 antibody or an anti-PD-L1 antibody capable of being administered to a human patient. In more specific embodiments, the anti-PD-1 antibody is nivolumab or pembrolizumab and the anti-PD-L1 antibody is atezolizumab, durvalumab, avelumab or CX-072 (CytomX Therapeutics).

The GnRH agonist can be any GnRH agonist such as, for example, leuprolide, buserelin, histrelin, goserelin, deslorelin, nafarelin, or triptorelin. The preferred GnRH agonist is triptorelin. Triptorelin is advantageously administered as a depot formulation. Most preferred is a one-month sustained-release formulation. The latter formulation is based on poly-(D,L-lactide-co-glycolide) (PLGA) microparticles of triptorelin acetate or of triptorelin pamoate and are designed to deliver 3 mg or 3.75 mg triptorelin, respectively.

The GnRH antagonist can be any GnRH antagonist such as, for example, cetrorelix, ganirelix, abarelix, degarelix, elagolix, relugolix, KLH-2109 and ASP-1707.

Preferred methods of treatment of the present disclosure involve biweekly (i.v.) administration of nivolumab at a dose of 3 mg/kg body weight or at a 240 mg flat dose and monthly (i.m.) administration of triptorelin, or triweekly (i.v.) administration of pembrolizumab at a dose of 2 mg/kg body weight or at a 200 mg flat dose and monthly (i.m.) administration of triptorelin.

To counteract the so-called “flare effect”, that is the initial peak of testosterone before its suppression, patients can be further administered an effective amount of an anti-androgen for two to four weeks beginning before or at the time of first administration of a GnRH agonist. Suitable anti-androgens are bicalutamide, cyproterone acetate, enzalutamide, apalutamide or darolutamide. Preferred is bicalutamide, which is administered daily at a 50 mg oral dose.

The methods of treatment disclosed herein may be administered to a treatment-naïve human patient or to a human patient who has previously been subjected to treatment including an anti-PD-1 molecule and has been found refractory or has become resistant to such treatment.

### DETAILED DESCRIPTION OF THE INVENTION

The present disclosure relates to a method of treating cancer patients with an anti-PD-1 molecule in association with a GnRH-A or a GnRH-At. Without wishing to be bond to any theory, the GnRH-A or GnRH-At is added to modify the immune system of the cancer patient, resulting in increasing the potential targets of the anti-PD-1 molecule (in particular the tumor infiltrating lymphocytes — TILs). As a consequence, the combination of GnRH-A or GnRH-At and anti-PD-1 molecule is expected to result in a better therapeutic effect compared with the individual active agents, without causing the severe immune-related toxicity that combinations of immune checkpoint inhibitors produce.

### Definitions

The term “in association with”, as used herein in reference to administration of an anti-PD-1 molecule with a GnRH-A or GnRH-At, means that the GnRH-A or GnRH-At is administered prior to, concurrently with, or after administration of the anti-PD-1 molecule. Within a therapeutic regimen, one of the active agents may be administered at shorter intervals than the other. Both agents may be administered independently by any suitable route, e.g., orally or parenterally, e.g., intramuscularly, intraperitoneally, subcutaneously or intravenously. Anti-PD-1 molecules are administered preferably by intravenous infusion, GnRH-A formulations are preferably administered, depending on the formulation, intramuscularly, subcutaneously or intranasally and GnRH-At formulations are preferably administered intramuscularly, subcutaneously or orally.

A “complete response” or “complete remission” or “CR” indicates the disappearance of all signs of tumor or cancer in response to treatment. This does not always mean the cancer has been cured. Complete response is generally measured using the RECIST v1.1 criteria. Eisenhauer, E. A., Eur. J. Cancer, 45: 228-47 (2009).

A “partial response” or “PR” refers to a decrease of at least 30% in the sum of the diameters of target lesions, in response to treatment. Eisenhauer, E. A., Eur. J. Cancer, 45: 228-47 (2009).

“Progressive disease” or “disease that has progressed” or “disease progression” refers to the appearance of one or more new lesions or tumors and/or the unequivocal progression of existing non-target lesions and/or at least a 20% increase in the sum of diameters of target lesions. Eisenhauer, E. A., Eur. J. Cancer, 45: 228-47 (2009).

“Stable disease” refers to disease without progression or relapse. In stable disease there is neither sufficient tumor diameter decrease to qualify for partial response nor sufficient tumor diameter increase to qualify as progressive disease. Eisenhauer, E. A., Eur. J. Cancer, 45: 228-47 (2009).

### Methods of Use and Pharmaceutical Compositions

The present disclosure relates to a method of treating cancer patients with an anti-PD-1 molecule in association with a GnRH-A or a GnRH-At.

Various sustained release formulations are available for different GnRH-A, mostly using polymeric drug delivery systems. For example, triptorelin formulations based on poly-(D,L-lactide-co-glycolide) (PLGA) microparticles: one-month triptorelin formulations delivering 3 mg of triptorelin (in acetate form) or 3.75 mg triptorelin (in pamoate form), 3-month triptorelin formulations delivering 11.25 mg triptorelin (in pamoate or acetate form), respectively, and a 6-month formulation delivering 22.5 mg triptorelin (in pamoate form) (Tradenames for triptorelin formulations include e.g. Decapeptyl, Trelstar, Pamorelin, Dipherelin). These formulations are provided as lyophilized powders that need to be suspended in an aqueous medium prior to intramuscular and/or subcutaneous injection. Also available are leuprolide acetate PLGA formulations 1, 3, 4 and 6 months, either in the form of microspheres (Lupron Depot) or of a liquid forming a depot after injection (Eligard). One-month formulations release either 3.75 mg, 7.5 mg, 11.25 mg or 15 mg leuprolide, 3-month formulations release 11.25 mg, 22.5 mg or 30 mg leuprolide, 4-month formulations release 30 mg leuprolide and 6 month formulations release 45 mg leuprolide. There are also implants of leuprolide acetate (Leuprorelin Sandoz) with a dose of 3.6 mg for one month and 5 mg for 3 months. “Suprefact Depot” formulations comprise buserelin acetate in PLGA. A two-month formulation contains the equivalent of 6.3 mg and a three-month formulation of 9.45 mg buserelin. These formulations are administered subcutaneously in a lateral abdominal region. “Vantas” and “Supprelin” contain 50 mg of histrelin (in acetate form) in a non-biodegradable hydrogel diffusion-controlled reservoir. These subcutaneous implants are intended for 12-month release of histrelin in a daily amount of 50-60 μg/day. “Zoladex” are PLGA-based implants of goserelin (present in acetate form), delivering 3.6 mg (one-month release) and 10.8 mg (three-month release) of goserelin, respectively. Synarvel is a nasal spray dispensing nafarelin (present as acetate). The spray delivers doses of 200 μg of nafarelin; the recommended dose is 400 μg per day.

Various formulations are available for different GnRH-At. In particular, non peptidic GnRH-At are administered orally (e.g. elagolix may be administered at 150 mg or 200 mg twice daily and relugolix may be administered at doses between 10 and 40 mg daily or between 80 and 160 mg daily) and peptidic GnRH-At are administered intramuscularly or subcutaneously. Degarelix formulations (“Firmagon”) are available as a sterile lyophilized powder for injection containing degarelix (as the acetate) and mannitol. For example, a starting dose comprises 240 mg given as two 3 mL subcutaneous injections of 120 mg each. Each vial of Firmagon 120 mg contains 120 mg degarelix and is to be reconstituted with a prefilled syringe containing 3 mL of Sterile Water for Injection to deliver 120 mg degarelix at a concentration of 40 mg/mL. A maintenance dose comprises 80 mg given as one 4 mL subcutaneous injection. Each vial of Firmagon 80 mg contains 80 mg degarelix that is to be reconstituted with a prefilled syringe containing 4.2 mL of Sterile Water for Injection—4 mL is withdrawn to deliver 80 mg degarelix at a concentration of 20 mg/mL. Maintenance doses are intended for monthly administrations. Available formulations of cetrorelix (“Cetrotide”) contain 0.25 mg or 3 mg cetrorelix (acetate) as a sterile lyophilized powder intended for subcutaneous injection after reconstitution with Sterile Water for Injection, that comes supplied in either a 1.0 mL (for 0.25 mg vial) or 3.0 mL (for 3 mg vial) pre-filled syringe. Each vial of Cetrotide 0.25 mg (multiple dose regimen, intended for daily administration) contains 0.26-0.27 mg cetrorelix acetate, equivalent to 0.25 mg cetrorelix, and 54.80 mg mannitol. Each vial of Cetrotide 3 mg (single dose regimen) contains 3.12-3.24 mg cetrorelix acetate, equivalent to 3 mg cetrorelix, and 164.40 mg mannitol. Ganirelix Acetate Injection is supplied as a colorless, sterile, ready-to-use, aqueous solution intended for subcutaneous administration. Each sterile, prefilled syringe contains 250 mcg/0.5 mL of Ganirelix Acetate, 0.1 mg glacial acetic acid, 23.5 mg mannitol, and water for injection adjusted to pH 5.0 with acetic acid, NF and/or sodium hydroxide, NF. Abarelix for injectable suspension (“Plenaxis”) is supplied as a white to off-white sterile dry powder, which, when mixed with the diluent, 0.9% Sodium Chloride Injection, USP, becomes a depot formulation intended for intramuscular injection. The single-dose vial contains 113 mg of anhydrous free base abarelix peptide (net) supplied in an abarelix Carboxymethylcellulose (CMC) complex. This complex contains 19.1 to 31 mg of CMC. After the vial is reconstituted with 2.2 mL of sodium chloride injection, 2 mL is administered to deliver a dose of 100 mg of abarelix (net) as the abarelix CMC complex at a pH of 5±1. Plenaxis may be administered intramuscularly on day 1, 15, 29 (week 4) and every 4 weeks thereafter.

Preferred anti-PD-1 molecules are anti-PD-1 antibodies such as nivolumab or pembrolizumab and anti-PD-L1 antibodies such as atezolizumab, durvalumab or avelumab. Nivolumab is being distributed under the brand “Opdivo”. It comes as a 10mg/ml solution that comprises the nivolumab antibody, mannitol, pentetic acid, polysorbate 80, sodium chloride, sodium citrate dihydrate and water. For administration, it is diluted into 0.9% sodium chloride or 5% dextrose. Pembrolizumab is being distributed under the brand “Keytruda”. It is furnished as a solid composition comprising 50 mg antibody and inactive ingredients L-histidine, polysorbate-80 and sucrose. For administration, the composition is suspended in 0.9% sodium chloride. Atezolizumab is being distributed under the brand “Tecentriq”. It is furnished as a liquid composition comprising 1200 mg antibody in 20 mL solution. Durvalumab is being distributed under the brand “Imfinzi”. It is furnished as a liquid composition comprising 500 mg/10mL (50 mg/mL) solution in a single-dose vial or 120 mg/2.4mL (50 mg/mL) solution in a single-dose vial.

Suitable doses of immune checkpoint inhibitors are those currently used in clinical practice. A suitable dose of nivolumab is 3 mg/kg body weight or a 240 mg flat dose. This dose is administered by intravenous infusion during a period of 60 min. A suitable dose of pembrolizumab is 2 mg/kg body weight. This dose is administered by intravenous infusion during a period of 30 min. A flat dose of 200 mg can also be administered. These doses may be adapted in parallel with adaptations accepted in clinical practice. Dosing of nivolumab is typically repeated every two weeks, and that of pembrolizumab every three weeks.

The GnRH-A is administered preferably as a sustained release formulation. Various suitable sustained release formulations were described above. Preferred formulations are microparticle formulations releasing triptorelin over one month and comprising a biologically active and well tolerated dose, such as about 1 mg, 1.5 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 5.75 mg, 6 mg, 6.5 mg, 6.75 mg, 7 mg, 7.5 mg, 7.75 mg or 8 mg of triptorelin. Such formulations can be obtained using the same process of preparation as that of a commercial one-month triptorelin formulation, and simply adjusting the amount of microparticles to obtain the desired quantity/dose of triptorelin. For example, approximately 4% (w/w) of triptorelin pamoate is mixed with approximately 96% (w/w) PLGA 50/50 having a viscosity of about 0,50 dL/g, at room temperature. The given mixture is duly homogenized, subjected to progressive compression and simultaneously to a progressive heating, before extrusion at a temperature of approximately 110° C. The extrudate is cut into pellets and ground at a temperature of about —100° C. The microgranules obtained after grinding are sieved below 106 micrometers. The appropriate quantity of microgranules is filled in vials, which are then lyophilized and further sterilized with gamma irradiation. The latter microparticle formulations are re-suspended with water prior to intramuscular administration of the resulting aqueous suspension. Typically, they will be re-administered after one month, two months, three months, etc., (or after 4 weeks, 8 weeks, 12 weeks, etc,.) until the end of treatment. The formulations will cause a decrease of gonadotropins and a fall in plasma testosterone and estradiol to levels causing substantial immunological modifications (in particular, increasing TILs), which levels are maintained until the end of the treatment period. It is understood that the triptorelin formulations may be substituted with sustained release formulations of other GnRH-A as described above, which formulations are designed to similarly reduce sex hormones.

Administration of GnRH-A is known to cause an initial increase in FSH and LH secretion, which results in an increased circulating testosterone (estrogen in women) level (the so-called “flare effect”). After about two to four weeks, a profound hypogonadal effect (i.e. decrease in testosterone and estrogen) is achieved through pituitary GnRH receptor downregulation by internalization of receptors followed by gonadal suppression. The treating physician may consider it beneficial to counteract this flare by co-administration of an androgen receptor antagonist for the period known to be required for the attainment of the sustained reduced levels of sex hormones causing substantial immunological modifications (in particular, increasing TILs), such as for example two to four weeks. A suitable androgen receptor antagonist is bicalutamide that is typically administered as a 50 mg/day oral dose. Alternatives are the older non-steroidal anti-androgens flutamide or nilutamide which are less potent, having lower affinities for androgen receptor as well as shorter half-lives than bicalutamide. Higher potency alternatives would include enzalutamide, apalutamide (ARN-509) or darolutamide (ODM-201). Yet other alternatives may be steroidal anti-androgens such as cyproterone acetate. Dosages for all mentioned anti-androgens are well known in the medical field.

The present disclosure relates to a method of treating human cancer patients with an anti-PD-1 molecule in association with a GnRH-A or GnRH-At. This method of treatment is suitable for all indications in which efficacy of an anti-PD-1 agent used as a single agent or in combination with another agent can be demonstrated. These indications can include all types of human cancer. In a particular embodiment, they include all types of human cancer except prostate cancer. In more particular embodiments, the indications are limited to melanoma, lung cancer, kidney cancer, bladder cancer, ovarian cancer, head and neck cancer, gastric cancer, colorectal cancer, triple-negative breast cancer, mesothelioma and Hodgkin's lymphoma.

The duration of combination treatment with an anti-PD-1 molecule, preferably an anti-PD-1 antibody such as nivolumab or pembrolizumab, in association with a GnRH-A or GnRH-At, preferably a triptorelin one-month sustained release formulation, will be determined by the treating physician based on the clinical benefit and tolerance.

Combination treatment of suitable cancers with an anti-PD-1 antibody such as nivolumab or pembrolizumab, in association with a GnRH-A such as triptorelin or a GnRH-At is expected to be more effective than treatment with the respective anti-PD-1 antibody alone. This is demonstrated in clinical experiments in which efficacy outcomes such as tumor response according to RECIST v. 1.1 (Eisenhauer, E. A., Eur. J. Cancer, 45: 228-47 (2009)), best overall response (BOR), duration of response, and objective response rate (ORR) are determined. Immune-related toxicities of the combination treatment are expected to be essentially the same as those resulting from treatment with the anti-PD-1 antibody alone.

The method of treatment disclosed herein involves administering by intravenous infusion 3 mg/kg body weight or a 240 mg flat dose of nivolumab (Opdivo) to a suitable cancer patient. The same dose is re-administered every two weeks. Dose amount and schedule of administration are as approved by regulatory agencies. Any modification of dose and schedule accepted by the medical community will also be applied to the presently described combination therapy. Another anti-PD-1 antibody may be used such as pembrolizumab (Keytruda). This antibody is also administered by infusion to a recommended dose of 2 mg/kg body weight or at a 200 mg flat dose. Re-dosing is every three weeks. Independently, the same patient is administered a sustained release formulation of a GnRH-A or a formulation of GnRH-At following the instructions of the manufacturer. From this formulation, GnRH-A or GnRH-At is released at a dose and/or rate sufficient to cause sex hormones to fall to levels causing substantial immunological modifications (in particular, increasing TILs) and to remain at these low levels to the end of the indicated period. A preferred slow-release GnRH-A formulation is a one-month triptorelin formulation, which formulation releases triptorelin at a rate sufficient to reduce sex hormones to levels causing substantial immunological modifications (in particular, increasing TILs). The formulation will be administered intramuscularly as a suspension comprising triptorelin-containing microparticles. Administration of triptorelin, GnRH-A or GnRH-At may be simultaneous with, prior to or subsequent to the administration of the anti-PD-1 antibody. It may be desirable that the first administration of GnRH-A sustained release formulation occurs considerably ahead of the anti-PD-1 antibody, for example by about three weeks, the time required for the sustained release formulation to reduce sex hormones to levels causing substantial immunological modifications (in particular, increasing TILs). Similarly, it may be desirable that the first administration of GnRH-At formulation occurs ahead of the anti-PD-1 antibody by the time required for the GnRH-At formulation to reduce sex hormones to levels causing substantial immunological modifications (in particular, increasing TILs). In addition or alternatively, the treating physician may opt to supplement the therapy with an anti-androgen to counteract the effect of the flare, for example during the two to four weeks starting before or following the initiation of GnRH-A. A preferred anti-androgen is bicalutamide (Casodex) that is typically given orally at 50 mg every day. Triptorelin one-month formulation may be re-administered after one month or 4 weeks.

The patients to whom the combination therapy disclosed herein is administered preferably are naïve patients, i.e., none of them will have received a therapy involving administration of an anti-PD-1 molecule. In another aspect, the combination therapy can be given to patients who failed or became resistant to therapy with an anti-PD-1 molecule (given either in monotherapy or in combination with (an) agent(s) different from GnRH-A or GnRH-At).

In other embodiments, the present disclosure also encompasses combinations of GnRH-A with other immune checkpoint inhibitors such as anti-CTLA-4 molecules.

### EXAMPLES

### Example 1: Phase I Study Debio 8200-IMM-101

To evaluate a combination therapy according to the present disclosure, a clinical trial is designed as an open label, single arm phase I study of the safety and efficacy of the combination of triptorelin and nivolumab (Debio 8200-IMM-101). The study population is male adult patients with refractory/relapsing locally advanced or metastatic histologically confirmed melanoma who progressed under anti-PD-1/PD-L1 (antibody)-containing regimens. Potentially evaluable patients are screened.

Fifteen evaluable patients are planned to be enrolled in the study. If any patient becomes unevaluable during the study, he is replaced.

The study involves 3 to 12 treatment cycles (depending on individual responses) of 28 days each. Patients receive triptorelin embonate (pamoate) 3.75 mg one-month formulation i.m. on day 1 of each 28-day cycle and nivolumab (Opdivo®) at 3 mg/kg body weight i.v. (1-hour infusion) on days 1 and 15 of each cycle. On day 1 of each cycle, triptorelin is administered prior to nivolumab. In addition, patients take bicalutamide 50 mg p.o. once daily for 28 days throughout cycle 1 only, to counteract the initial testosterone “flare” induced by triptorelin.

Tumor biopsies are taken prior to the beginning of dosing and at the end of three treatment cycles. The investigator then decides upon treatment continuation in the best interest of the patient, mainly based on the patient's clinical status and the tumor size assessment according to RECIST v1.1 at week 11 or 12, but the patient's overall disease evolution since diagnosis and initiation of anti-PD-1/PD-L1 treatment is also taken into account. Patients demonstrating benefit, i.e. complete response (CR), partial response (PR) or stable disease (SD) after 3 cycles, continue treatment for up to 12 cycles until disease progression, unacceptable toxicity (by investigator judgment), withdrawal of consent, or premature termination of the study, whichever comes first. In the absence of disease progression, patients undergo regular reevaluation of response every 3 cycles. Patients with disease progression after 3 cycles are reassessed 7-8 weeks later, i.e. at week 19 or 20. If disease progression is confirmed, they discontinue study treatment; otherwise they may continue treatment for up to 12 cycles at the discretion of the treating physician, as stated above.

Tumors are assessed according to RECIST version 1.1 guidelines by physical examination and photography with caliper, or CT scan (Computed Tomography) or MRI (Magnetic Resonance Imaging), performed as per site standard but whenever possible using contrast agent, at screening and during treatment at cycle 3 (week 11 or 12) and then every 3 cycles for up to 12 cycles (weeks 23, 35, and 49) until disease progression/end of treatment.

The primary endpoint is the incidence and severity of treatment-emergent adverse events/serious adverse events (AEs/SAEs), graded according to NCI-CTCAE version 4.03 criteria (as published on Jun. 14, 2010 by the U.S. Department of Health and Human Services, National Institutes of Health and National Cancer Institute) throughout the study. The most important secondary efficacy endpoints are (1) tumor response according to RECIST v1.1, (2) best overall response (BOR), (3) duration of response, and (4) objective response rate (ORR).

(1) Tumor response is assessed according to RECIST version 1.1. Complete responses (CR) and partial responses (PR) must be demonstrated by objective tumor assessment (CT scan or MRI) while disease progression may be determined clinically, based on the investigator's assessment.
(2) BOR is the best response (CR, PR, stable disease or disease progression) recorded from the start of study treatment until disease progression/recurrence is documented, a new systemic therapy is started or analysis cut-off, whichever occurs first.
(3) Duration of response is the time from documentation of tumor response to first documented evidence of disease progression.
(4) ORR is derived as any PR or CR recorded from the start of study treatment until disease progression/recurrence is documented or confirmed, a new systemic therapy is started or analysis cut-off, whichever occurs first.

Per protocol, to exclude pseudo-progressions, any disease progression observed according to RECIST v1.1 is confirmed as per iRECIST criteria (Seymour, L., et al., iRECIST: guidelines for response criteria for use in trials testing immunotherapeutics. Lancet Oncol, 2017. 18(3): p. e143-e152) between 4 weeks and 8 weeks after first observation.

The study is expected to show that the incidence and severity of treatment-emergent AEs/SAEs produced by the combination treatment is not worse than that observed for single therapy with nivolumab in terms of immune-related toxicity. Furthermore, the study is expected to show evidence of efficacy for the combination treatment by the measures of efficacy defined above.

### Example 2: Results of Combination Therapy on Patient A

A 71-year-old (at screening on 23 May 2018) male patient of White race had been operated on 26 Sep. 2016 for left lung lobectomy for melanoma metastasis of unknown origin. On 21 Aug. 2017, biopsy of PET-CT (Positron emission tomography—computed tomography) detected a new melanoma metastasis in the pancreas. From 27 Sep. 2017 until 13 Mar. 2018 (last administration at Cycle 9) he was treated with anti-PD-1 molecule pembrolizumab. On 12 Dec. 2017 a Partial Response (PR) was objectivized. However, on 30 Mar. 2018 PET-CT showed progression (PD) of the metastasis. On 23 May 2018, the size of this unique pancreatic metastasis was 34 mm on CT-scan.

On 12 Jun. 2018 the patient was enrolled in the protocol Debio 8200-IMM-101 and received the first injections of triptorelin -anti-androgen bicalutamide was added for 28 days- and anti-PD-1 molecule nivolumab.

As expected due to triptorelin treatment, the serum testosterone levels of the patient decreased from a baseline (Day 1) level of 15.7 nmol/L on 12 Jun. 2018 to a very low castrate level of 0.6 nmol/L four weeks later 10 Jul. 2018 (Day 29), and have remained in the castrate range (i.e. <1.735 nmol/L) until the last value available to date of 0.69 nmol/L on 29 Oct. 2018 (Day 141).

On 21 Aug. 2018, the tumor was stable at 31 mm on CT-scan. On 13 Nov. 2018, that is 5 months after treatment initiation, new CT-scan objectivized PR with 61% reduction of the tumor mass to 13 mm (compared to baseline of 34 mm). The PR was further confirmed by CT-scan on 12 Dec. 2018.

Immunohistochemistry on biopsy taken on 31 Aug. 2018 compared with baseline biopsy taken on 19 Apr. 2018 (i.e. 16 days after last pembrolizumab administration, end of cycle 9), demonstrated a 5-fold increase in number of CD8+TILs from 55/mm² to 300/mm².

Thus, the chemical castration with GnRH-A triptorelin associated with anti-PD-1 molecule nivolumab allowed the patient to develop an immunological boost translating into a local increase in number of TILs. The objective response obtained strongly suggests that the combination therapy of the present invention has rendered the patient responsive again to anti-PD-1 treatment, thus improving the patient outcome.

Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as reference to an element or elements is intended to provide support for a similar aspect or embodiment of the invention that “consists of”,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

1. A method of treatment of a human patient having a cancer, the method comprising administering to the patient an effective dose of an anti-PD-1 molecule in association with an effective dose of a GnRH agonist or a GnRH antagonist.

2. The method of treatment of claim 1, wherein the cancer is any cancer except a prostate cancer.

3. The method of treatment of claim 1, wherein the cancer is selected from a melanoma, a lung cancer, a kidney cancer, a bladder cancer, an ovarian cancer, a head and neck cancer, a gastric cancer, a colorectal cancer, a triple-negative breast cancer, a mesothelioma and a Hodgkin's lymphoma.

4. The method of treatment of claim 3, wherein the cancer is a melanoma.

5. The method of treatment of claim 1, wherein the anti-PD-1 molecule is an anti-PD-1 antibody or an anti-PD L1 antibody.

6. The method of treatment of claim 5, wherein the anti-PD-1 molecule is nivolumab.

7. The method of treatment of claim 5, wherein the anti-PD-1 molecule is pembrolizumab.

8. The method of treatment of claim 5, wherein the anti-PD-1 molecule is atezolizumab.

9. The method of treatment of claim 5, wherein the anti-PD-1 molecule is durvalumab.

10. The method of treatment of claim 5, wherein the anti-PD-1 molecule is avelumab.

11. The method of treatment of claim 1, wherein the anti-PD-1 molecule is associated with an effective dose of a GnRH agonist.

12. The method of treatment of claim 11, wherein the GnRH agonist is leuprolide, buserelin, histrelin, goserelin, deslorelin, nafarelin or triptorelin.

13. The method of treatment of claim 1, wherein the anti-PD-1 molecule is associated with an effective dose of a GnRH antagonist.

14. The method of treatment of claim 13, wherein the GnRH antagonist is cetrorelix, ganirelix, abarelix, degarelix, elagolix, relugolix, KLH-2109 or ASP-1707.

15. The method of treatment of claim 12, wherein the GnRH agonist is triptorelin.

16. The method of treatment of claim 15, wherein triptorelin is administered in the form of a one-month sustained-release formulation.

17. The method of treatment of claim 16, wherein the anti-PD1 molecule is nivolumab, the nivolumab is administered biweekly at a dose of 3 mg/kg body weight or at 240 mg flat dose, and the triptorelin is administered once every month.

18. The method of treatment of claim 16, wherein the anti-PD1 molecule is pembrolizumab, the pembrolizumab is administered triweekly at a dose of 2 mg/kg body weight or at 200 mg flat dose, and the triptorelin is administered once every month.

19. The method of treatment of claim 11, wherein the patient is further administered an effective amount of an anti-androgen during a period of two to four weeks, said period beginning at the time of first administration of the GnRH agonist.

20. The method of treatment of claim 19, wherein the anti-androgen is bicalutamide, cyproterone acetate, enzalutamide, apalutamide or darolutamide.

21. The method of treatment of claim 1, wherein the human patient has previously been subjected to treatment including an anti-PD-1 molecule and has been found refractory or has become resistant to such treatment.

22-43. (canceled)
